(19) United States
(12) Patent Application Publication
(10) Pub. No.: US 2021/0052251 A1
(43) Pub. Date: Feb. 25, 2021

(54) METHODS AND APPARATUSES FOR GUIDING A USER TO COLLECT ULTRASOUND DATA

(71) Applicant: Butterfly Network, Inc., Guilford, CT (US)

(72) Inventors: Nathan Silberman, Brooklyn, NY (US); Igor Lovchinsky, New York, NY (US); Tomer Gafner, Forest Hills, NY (US)

(73) Assignee: Butterfly Network, Inc., Guilford, CT (US)

(21) Appl. No.: 17/000,227

(22) Filed: Aug. 21, 2020

Related U.S. Application Data
(60) Provisional application No. 62/891,251, filed on Aug. 23, 2019.

(51) Int. Cl. A61B 8/08; A61B 8/00

(52) U.S. Cl. CPC A61B 8/5207; A61B 8/469; A61B 8/5223; A61B 8/4245

(57) ABSTRACT

Aspects of the technology described herein relate to incorporating how a user has previously moved an ultrasound device when providing instructions for moving the ultrasound device. Some embodiments include providing a first instruction for moving the ultrasound device, determining a difference between the first instruction and a movement of the ultrasound device, determining a second instruction for moving the ultrasound device based on the difference between the first instruction and the movement of the ultrasound device, and providing the second instruction for moving the ultrasound device. Some embodiments include providing a notification to the user regarding the user not following an instruction accurately. Some embodiments include providing a first instruction among a set of instructions that also includes a second instruction that has not yet been provided. Based on determining that the user did not follow the first instruction accurately, the second instruction is provided.

### CROSS-REFERENCE TO RELATED APPLICATIONS

This application claims the benefit under 35 U.S.C. § 119(e) of U.S. Patent Application Ser. No. 62/891,251, filed Aug. 23, 2019 under Attorney Docket No. B 1348.70160US00, and entitled “METHODS AND APPARATUSES FOR GUIDING A USER TO COLLECT ULTRASOUND DATA,” which is hereby incorporated by reference herein in its entirety.

### FIELD

Generally, the aspects of the technology described herein relate to collection of ultrasound data. Certain aspects relate to guiding a user to collect ultrasound data.

### BACKGROUND

Ultrasound devices may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the device at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect of the application, an apparatus comprises a processing device in operative communication with an ultrasound device, the processing device configured to provide a first instruction for moving the ultrasound device; determine a difference between the first instruction and a movement of the ultrasound device; determine, based on the difference between the first instruction and the movement of the ultrasound device, a second instruction for moving the ultrasound device; and provide the second instruction for moving the ultrasound device.

In some embodiments, the processing device is configured, when determining the difference between the first instruction and the movement of the ultrasound device, to determine the movement of the ultrasound device by determining motion and/or orientation of the ultrasound device relative to the processing device. In some embodiments, the processing device is configured, when determining the difference between the first instruction and the movement of the ultrasound device, to determine the movement of the ultrasound device by using one or more of images/video from the processing device, motion and/or orientation data from the processing device, and motion and/or orientation data from the ultrasound device to determine the movement of the ultrasound device relative to the processing device.

In some embodiments, the first instruction indicates a direction for moving the ultrasound device, the movement data describes a direction that the ultrasound device has moved, and the processing device is configured, when determining the difference between the first instruction and the movement of the ultrasound device, to determine a difference between the direction for moving the ultrasound device and the direction that the ultrasound device has moved. In some embodiments, the processing device is configured, when determining the second instruction for moving the ultrasound device, to determine the second instruction such that the second instruction compensates for the difference between the first instruction and the movement of the ultrasound device.

In some embodiments, the processing device is configured, when determining the second instruction for moving the ultrasound device, to subtract the difference between the first instruction and the movement of the ultrasound device from a direction in which the processing device determines the ultrasound device should be moved. In some embodiments, the processing device is configured to provide the first instruction and the second instruction as part of a single scan. In some embodiments, the processing device is configured to provide the first instruction and the second instruction for moving the ultrasound device to a target pose. In some embodiments, the processing device is configured to provide the first instruction for moving the ultrasound device to a first target pose, and to provide the second instruction for moving the ultrasound device to a second target pose. In some embodiments, the first target pose is a pose where the ultrasound device can collect ultrasound data from one anatomical region, and the second target pose is a pose where the ultrasound device can collect ultrasound data from a second anatomical region. In some embodiments, the first and second anatomical regions are anatomical regions scanned as a part of an imaging protocol. In some embodiments, the processing device is configured to provide the first instruction and the second instruction as part of different scans. In some embodiments, the processing device is configured to store in memory the difference between the first instruction and the movement of the ultrasound device from scan to scan and use that difference for providing the second instruction in a subsequent scan. In some embodiments, the processing device is configured to compute an average of differences between instructions provided and subsequent movements of the ultrasound device across multiple scans, and to use the average of differences to provide the second instruction.

According to another aspect of the application, an apparatus comprises a processing device in operative communication with an ultrasound device, the processing device configured to provide an instruction for moving the ultrasound device; determine that a user did not follow the instruction accurately; and based on determining that the user did not follow the instruction accurately, provide a notification to the user regarding the user not following the instruction accurately.

In some embodiments, the notification communicates to the user that the user should carefully follow instructions. In some embodiments, the processing device is configured, when providing the notification, to highlight graphical directions. In some embodiments, the notification indicates to the user that the user can access a help page to obtain a refresher on how to use the processing device.

According to another aspect of the application, an apparatus comprises a processing device in operative communication with an ultrasound device, the processing device configured to provide a first instruction among a set of instructions for moving the ultrasound device, where the set of instructions includes the first instruction and a second instruction, and the second instruction has not yet been provided; determine that a user did not follow the first instruction accurately; and based on determining that the user did not follow the first instruction accurately, provide the second instruction.

In some embodiments, the processing device is configured to cease to provide the first instruction before the user has accurately followed the first instruction.

Some aspects include at least one non-transitory computer-readable storage medium storing processor-executable instructions that, when executed by at least one processor, cause the at least one processor to perform the above aspects and embodiments. Some aspects include a method to perform the actions that the apparatus is configured to perform.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1 is an illustration of an instruction for moving an ultrasound device, in accordance with certain embodiments described herein;

FIG. 2 is an illustration of a subject, an ultrasound device, and a path traveled by the ultrasound device relative to the subject, in accordance with certain embodiments described herein;

FIG. 3 is an illustration of another instruction for moving an ultrasound device, in accordance with certain embodiments described herein;

FIG. 4 is an illustration of another instruction for moving an ultrasound device, in accordance with certain embodiments described herein;

FIG. 5 is a flow diagram of a process for guiding a user to collect ultrasound data, in accordance with certain embodiments described herein;

FIG. 6 is another flow diagram of a process for guiding a user to collect ultrasound data, in accordance with certain embodiments described herein;

FIG. 7 is another flow diagram of a process for guiding a user to collect ultrasound data, in accordance with certain embodiments described herein; and

FIG. 8 illustrates a schematic block diagram of an example ultrasound system upon which various aspects of the technology described herein may be practiced.

### DETAILED DESCRIPTION

Conventional ultrasound systems are large, complex, and expensive systems that are typically only purchased by large medical facilities with significant financial resources. Recently, cheaper and less complex ultrasound devices have been introduced. Such devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application), and published as U.S. Pat. Pub. No. 2017-0360397-A1, which is incorporated by reference herein in its entirety. The reduced cost and increased portability of these new ultrasound devices may make them significantly more accessible to the general public than conventional ultrasound devices.

The inventors have recognized and appreciated that although the reduced cost and increased portability of ultrasound devices makes them more accessible to the general populace, people who could make use of such devices have little to no training for how to use them. Ultrasound examinations often include the acquisition of ultrasound images that contain a view of a particular anatomical structure (e.g., an organ) of a subject. Acquisition of these ultrasound images typically requires considerable skill. For example, an ultrasound technician operating an ultrasound device may need to know where the anatomical structure to be imaged is located on the subject and also how to properly position the ultrasound device on the subject to capture a medically relevant ultrasound image of the anatomical structure. Holding the ultrasound device a few inches, centimeters, or millimeters too high or too low on the subject may make the difference between capturing a medically relevant ultrasound image and capturing a medically irrelevant ultrasound image. As a result, non-expert operators of an ultrasound device may have considerable trouble capturing medically relevant ultrasound images of a subject. Common mistakes by these non-expert operators include capturing ultrasound images of the incorrect anatomical structure and capturing foreshortened (or truncated) ultrasound images of the correct anatomical structure.

Accordingly, the inventors have developed technology for guiding an operator of an ultrasound device how to move the ultrasound device in order to capture medically relevant ultrasound data. To guide the user, a processing device may output one or more instructions for moving the ultrasound device from a current position and orientation to the target position and orientation. To output an instruction, the processing device may capture, using a camera, a video in real-time of the ultrasound device and/or the subject, and display an augmented reality display including a directional indicator (e.g., an arrow) superimposed on the video, where the directional indicator indicates the instruction for moving the ultrasound device. For example, if the instruction is to move the ultrasound device towards the subject's head, the processing device may display a directional indicator towards the subject. As another example, to output an instruction, the processing device may output text or audio.

The inventors have recognized that a user may not always follow instructions correctly, which may hamper their ability to collect ultrasound data as instructed by the processing device. The inventors have recognized that a processing device may track how a user is actually following instructions and incorporate this information when providing future instructions. As one example of how a processing device may incorporate such information when providing future instructions, in some embodiments, the processing device may adjust instructions to compensate for the past inaccurate movement of the ultrasound device by the user. As another example, the processing device may provide an instruction (e.g., with printed text or highlighted arrows) to the user to carefully follow the instructions and/or indicate to the user that the user may access a help page to obtain a refresher on how to use the processing device. As another example, the processing device may provide sets of instructions that avoid instructions the user has not followed well before.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

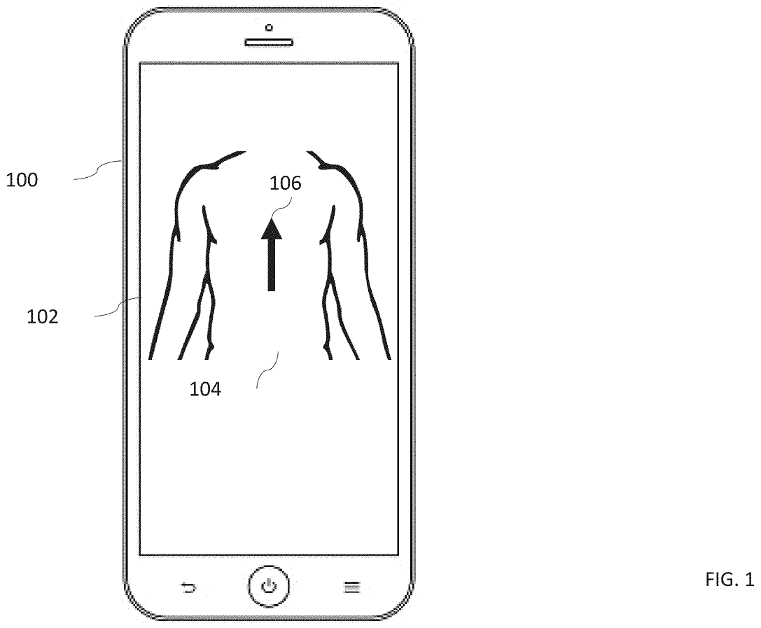

FIGS. 1-4 illustrate one example of how a processing device may incorporate how a user has previously moved an ultrasound device when providing instructions for moving the ultrasound device. FIG. 1 is an illustration of an initial instruction 106 for moving an ultrasound device, in accordance with certain embodiments described herein. FIG. 1 illustrates a processing device 100. The processing device 100 includes a display screen 102. The display screen 102 depicts an image 104 of a subject and the initial instruction 106. The processing device 100 may be in operative communication with an ultrasound device. For example, the processing device 100 may be a mobile phone, tablet, or laptop. The ultrasound device and the processing device 100 may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). The image 104 of the subject may be a real-time image of the subject, in other words, a frame of a video of the subject collected in real time (e.g., by a camera on the processing device) or a static image of a subject (e.g., a photograph of a subject who may or may not be the same as the actual subject, or a cartoon/stylized image of a subject). The initial instruction 106 may be an instruction for moving the ultrasound device relative to the subject in order to collect ultrasound data. The processing device may have determined, based on the current position of the ultrasound device, that the ultrasound device should be moved as indicated by the initial instruction 106 in order to collect particular ultrasound data (e.g., an ultrasound image depicting a particular anatomical view). In FIG. 1, the initial instruction 106 is a directional indicator, in particular an arrow, that is overlaid on the image 104 of the subject and depicts the direction relative to the subject that the ultrasound device should be moved in order to collect the ultrasound data. However, there may be other forms for the initial instruction 106, such as a marker at the location on the image 104 of the subject where the ultrasound device should be moved, or text describing how the ultrasound device should be moved. In FIG. 1, the initial instruction 106 points straight towards the subject's head, however the initial instruction 106 may point in other directions depending on the ultrasound data to be collected and the current location of the ultrasound device.

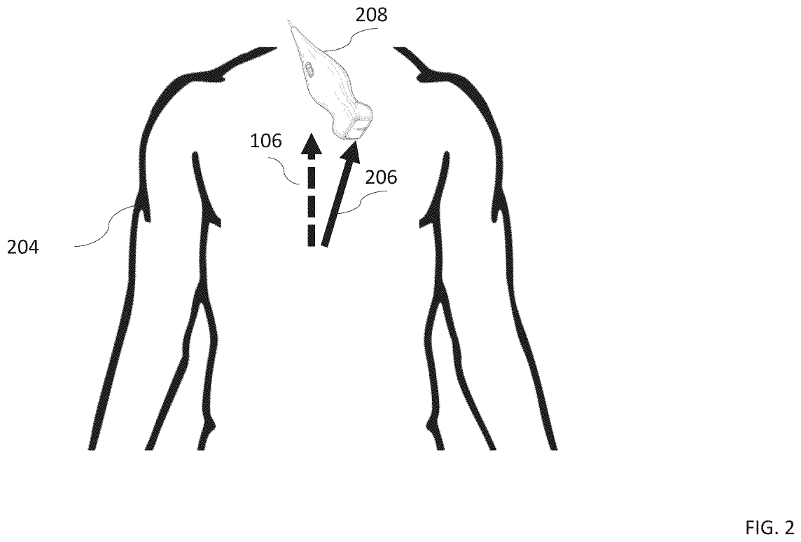

FIG. 2 is an illustration of a subject 204, an ultrasound device 208, and a path 206 traveled by the ultrasound device 208 relative to the subject 204, in accordance with certain embodiments described herein. A user may have moved the ultrasound device 208 along the path 206 in response to the initial instruction 106 displayed by the processing device 100 in FIG. 1. As can be seen, the actual path 206 traveled by the ultrasound device 208 is different from the direction for moving the ultrasound device 208 that is indicated by the initial instruction 106. In particular, the actual path 206 is directed slightly to the right of straight towards the head of the subject 204 instead of straight towards the subject's head in FIG. 1.

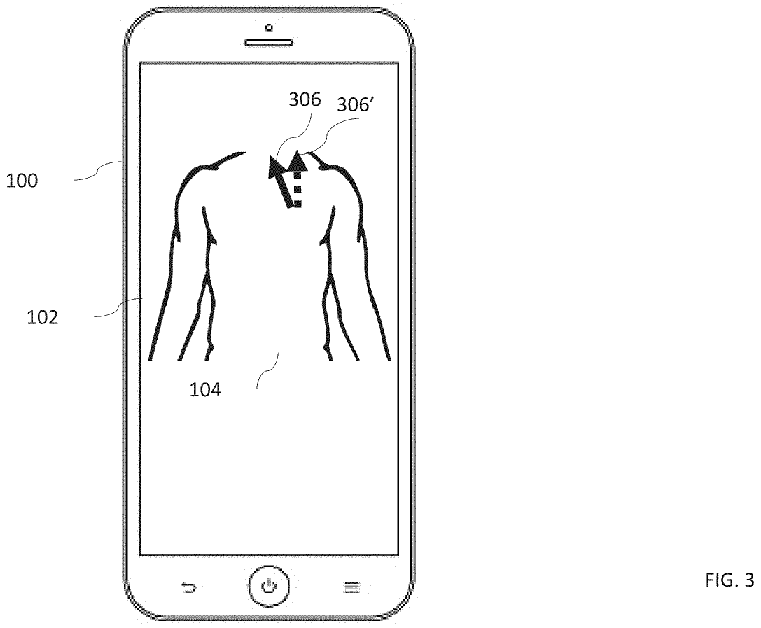

FIG. 3 is an illustration of a corrective instruction 306 for moving the ultrasound device 208, in accordance with certain embodiments described herein. The processing device 100 may have generated the corrective instruction 306 based on the difference between the actual path 206 traveled by the ultrasound device 208 and the initial instruction 106. In other words, the corrective instruction 306 may compensate for the difference between the path 206 traveled by the ultrasound device 208 and the initial instruction 106. In the example of FIG. 3, it may be assumed that the processing device 100 determined, based on the current position of the ultrasound device 208, that the ultrasound device 208 should now be moved straight towards the subject 204's head (i.e., the instruction should be the instruction 306′) in order to collect particular ultrasound data (e.g., an ultrasound image depicting a particular anatomical view). However, based on the difference between the actual path 206 traveled by the ultrasound device 208 and the initial instruction 106, the processing device 100 may have generated and displayed the corrective instruction 306 such that it is directed slightly to the left of straight towards the head of the subject 204. This may be helpful, because if the user tends to move the ultrasound device 208 more towards the right of the subject 204 than an instruction actually indicates (as the user initially did when moving the ultrasound device on the path 206 in response to the initial instruction 106), the processing device 100 may display an instruction more towards the left of the subject 204 then actually necessary in order to compensated for the user's inclination to unconsciously move the ultrasound device 208 slightly more towards the right of the subject 204 than the corrective instruction 306 indicates. This may mean that in response to the corrective instruction 306, the user maybe more likely to move the ultrasound device 208 substantially straight towards the head of the subject 204, which is assumed in this example to be the direction that the processing device 100 determines is necessary to collect particular ultrasound data. In other words, the processing device 100 may determine that the ultrasound device 108 should be moved in one direction (indicated by the instruction 306′) relative to the subject 204 but actually generate and display the corrective instruction 306 indicating a different direction, based on the previous difference between the instruction 106 and the path 206 traveled by the ultrasound device 108 (i.e., the user incorrectly following the initial instruction 106).

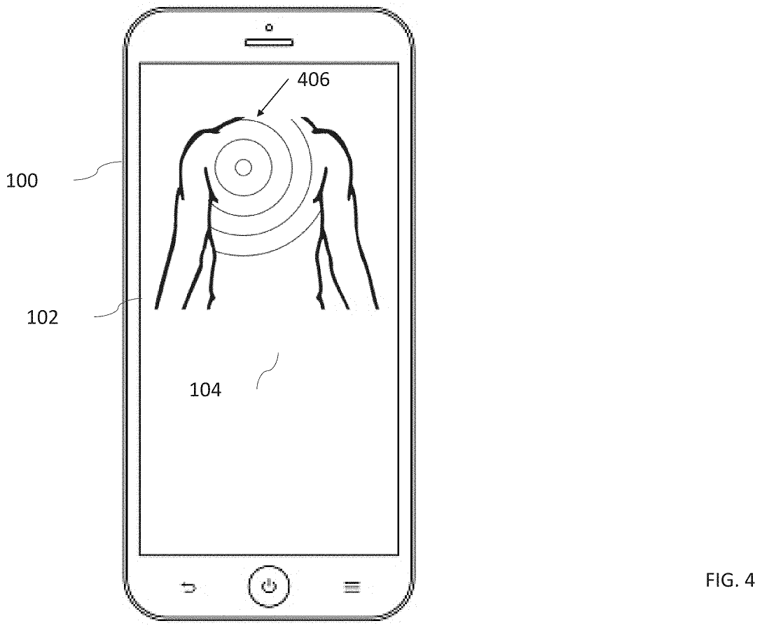

FIG. 4 is an illustration of another instruction 406 for moving the ultrasound device 208, in accordance with certain embodiments described herein. In FIG. 4, the instruction 406 includes concentric circles centered on the location to which the ultrasound device 208 should be moved. In some embodiments, there may be multiple sets of concentric circles, each set centered on a different location. For example, the different locations may be locations that are scanned as part of an imaging protocol (e.g., FAST, eFAST, or RUSH). In some embodiments, the size of the innermost circle may be based on how precise the scanning location must be. For example, cardiac imaging may require scanning a specific location, and therefore the innermost circle may be smaller. Lung imaging may not require scanning as specific a location, and therefore the innermost circle may be larger. As in FIG. 3, the instruction 406 may be based on the difference between a path traveled by the ultrasound device 208 and a previous instruction.

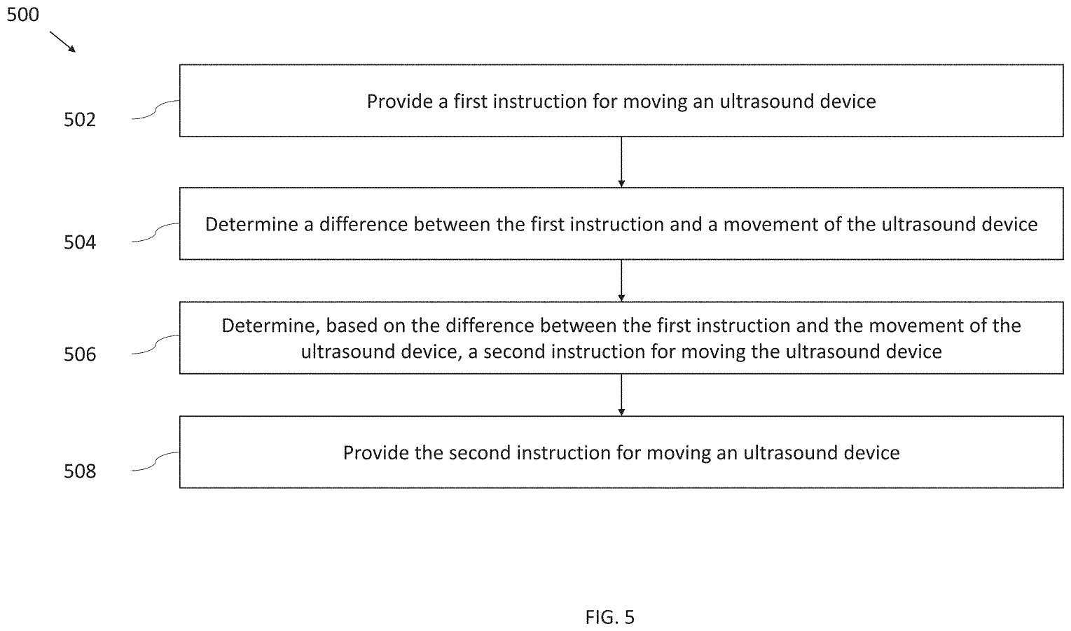

FIG. 5 is a flow diagram of a process 500 for how a processing device may incorporate how a user has previously moved an ultrasound device when providing instructions for moving the ultrasound device, in accordance with certain embodiments described herein. The process 500 is performed by a processing device in operative communication with an ultrasound device. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself may perform the process 500.

In act 502, the processing device provides a first instruction for moving the ultrasound device. In some embodiments, the processing device may input to a statistical model ultrasound data collected by the ultrasound device from a subject at the ultrasound device's current position. The statistical model may be configured to accept ultrasound data and output an instruction for moving the ultrasound device based on the ultrasound data. The first instruction may include an instruction for moving the ultrasound device in a particular direction relative to the subject in order to reach a target pose (i.e., position and/or orientation) relative to the subject being imaged and may include any combination of instructions to translate, rotate, and tilt the ultrasound device. For example, the first instruction may be to move the ultrasound device straight towards the head of the subject, which if the subject is standing may be equivalent to an instruction to move the ultrasound device along the axis of gravity in the upwards direction. The pose of the ultrasound device may be a pose of the ultrasound device relative to the subject such that the ultrasound device can collect an ultrasound image depicting a target anatomical view (e.g., a parasternal long axis view of the heart).

In some embodiments, the statistical model may be configured through training to accept ultrasound data and output an instruction for moving the ultrasound device to a target pose based on the ultrasound data. In particular, the statistical model may be trained on sets of training data, where each set of training data includes ultrasound data collected from a subject when the ultrasound device is at a particular pose relative to a subject, and a label indicating an instruction for moving the ultrasound device from the particular pose to the target pose. The training data may be labeled manually by an annotator (e.g., a doctor, sonographer, or other medical professional). The statistical model may thereby learn what instruction to provide for moving an ultrasound device from its current pose to a target pose based on inputted ultrasound data collected from the ultrasound device at its current pose. The statistical model may be a convolutional neural network, a random forest, a support vector machine, a linear classifier, and/or any other statistical model.

In some embodiments, the statistical model may be stored in memory on the processing device and accessed internally by the processing device. In other embodiments, the statistical model may be stored in memory on another device, such as a remote server, and the processing device may transmit the motion/and/or orientation data and the ultrasound data to the external device. The external device may input the ultrasound data to the statistical model and transmit the instruction outputted by the statistical model back to the processing device. Transmission between the processing device and the external device may be over a wired communication link (e.g., over Ethernet, a Universal Serial Bus (USB) cable or a Lightning cable) or over a wireless communication link (e.g., over a BLUETOOTH, WiFi, or ZIGBEE wireless communication link).

In some embodiments, providing the first instruction may include displaying a directional indicator, such as an arrow, that is overlaid on an image of a subject and depicts the direction relative to the subject that the ultrasound device should be moved in order to reach the target pose. The image of the subject may be a real-time image of the subject, in other words, a frame of a video of the subject collected in real time (e.g., by a camera on the processing device) or a static image of a subject (e.g., a photograph of a subject who may or may not be the same as the actual subject, or a cartoon/stylized image of a subject). In some embodiments, providing the first instruction may include displaying a marker at the location on the image of the subject where the ultrasound device should be moved, or text describing how the ultrasound device should be moved. The process 500 proceeds from act 502 to act 504.

In act 504, the processing device determines a difference between the first instruction and a movement of the ultrasound device. To determine the movement of the ultrasound device, the processing device may determine motion and/or orientation of the ultrasound device relative to the processing device. As will be described further below, the processing device may use one or more of images/video from the processing device, motion and/or orientation data from the processing device, and motion and/or orientation data from the ultrasound device to generate movement data that describes movement of the ultrasound device by a user relative to the processing device in response to the instruction provided in act 502. The processing device may compare the movement of the ultrasound device relative to the processing device and the first instruction as displayed by the processing device to determine the difference between the first instruction and the movement of the ultrasound device. For example, if the first instruction indicates a direction for moving the ultrasound device, and the movement data describes a direction that the ultrasound device has moved, then the processing device may determine a difference between the two directions. In some embodiments, the processing device may compute an average of the movement data to determine a single average direction describing how the ultrasound device has moved, and compare this single average direction to the direction indicated by the first instruction. As a specific example, if the first instruction indicates a direction that is parallel to the axis of gravity in the upwards direction, and the movement data describes a direction that is rotated 10 degrees clockwise from the axis of gravity in the upwards direction, then the processing device may determine a 10 degree clockwise difference between the first instruction and the movement of the ultrasound device. The process 500 proceeds from act 504 to act 506.

In act 506, the processing device determines, based on the difference between the first instruction and the movement of the ultrasound device that was determined in act 504, a second instruction for moving the ultrasound device. In the same manner as described with reference to act 502, the processing device may determine that the ultrasound device should be moved in a particular direction in order to reach a target pose from which the ultrasound device may collect an ultrasound image depicting a target anatomical view (e.g., a parasternal long axis view of the heart). However, the processing device may not provide an instruction to move the ultrasound device in this direction. Instead, the processing device may generate, based on the difference between the first instruction and the movement of the ultrasound device, an instruction for moving the ultrasound device that compensates for the difference between the first instruction and the movement of the ultrasound device. In some embodiments, the processing device may subtract the difference between the first instruction and the movement of the ultrasound device from the direction in which the processing device determines the ultrasound device should be moved. For example, if the processing device determines that the ultrasound device should be moved along the axis of gravity in the upwards direction, and the difference between the first instruction and the movement of the ultrasound device is 10 degrees clockwise, then the processing device may determine that the second instruction should be 10 degrees counterclockwise from the axis of gravity in the upwards direction. The process 500 proceeds from act 506 to act 508.

In act 508, the processing device provides the second instruction for moving the ultrasound device that was determined in act 506. Further description of providing instructions may be found with reference to act 502.

In some embodiments, the processing device may provide the first instruction and the second instruction as part of a single scan. For example, both may be instructions for moving the ultrasound device to a target pose. The processing device may provide the first instruction for moving the ultrasound device to a target pose at act 502, and based on the difference between the first instruction and the movement of the ultrasound device as determined at act 504, the processing device may modify the first instruction in real-time by providing the second instruction at act 508 for moving the ultrasound device to the target pose. As another example, the processing device may provide the first instruction for moving the ultrasound device to a first target pose at act 502, and based on the difference between the first instruction and the movement of the ultrasound device as determined at act 504, the processing device may provide the second instruction at act 508 for moving the ultrasound device to a second target pose. The first target pose may be a pose where the ultrasound device may collect ultrasound data from one anatomical region, and the second target pose may be a pose where the ultrasound device may collect ultrasound data from another anatomical region. The two anatomical regions may be anatomical regions scanned as a part of an imaging protocol, such as the FAST (focused assessment with sonography of trauma) imaging protocol. In some embodiments, the first and second instructions may be provided as part of different scans. For example, the processing device may store in memory the difference between the first instruction and the movement of the ultrasound device as determined in act 508 from scan to scan. The processing device may then use that difference for providing instructions (e.g., the second instruction at act 508) in subsequent scans. In some embodiments, the processing device may compute an average of differences between instructions provided and subsequent movements of the ultrasound device across multiple scans, and use that average difference to provide subsequent instructions (e.g., the second instruction at act 508). In some embodiments, the information about how the user previously moved the ultrasound device may be associated with a user profile, and the processing device may access this information when a user logs into his/her profile.

As described with reference to act 504, the processing device may determine motion and/or orientation of the ultrasound device relative to the processing device. This may include determining changes in position and/or changes in orientation. In some embodiments, the processing device may determine, based on video collected by the processing device that depicts the ultrasound device, a position of the ultrasound device relative to the processing device. The video may be collected by a camera on the processing device. In some embodiments, a user may hold the ultrasound device in one hand and hold the processing device in the other hand such that the ultrasound device is in view of the camera on the processing device. In some embodiments, a user may hold the ultrasound device in one hand and a holder (e.g., a stand having a clamp for holding the processing device) may hold the processing device such that the ultrasound device is in view of the camera on the processing device.

In some embodiments, a statistical model may be trained to determine the position of the ultrasound device relative to the processing device. In some embodiments, the statistical model may be trained as a keypoint localization model with training input and output data. Multiple images of the ultrasound device may be inputted to the statistical model as training input data. As training output data, an array of values that is the same size as the inputted image may be inputted to the statistical model, where the pixel corresponding to the location of the tip of the ultrasound device (namely, the end of the ultrasound device opposite the sensor portion) in the image is manually set to a value of 1 and every other pixel has a value of 0. Based on this training data, the statistical model may learn to output, based on an inputted image (e.g., a frame of the video of the ultrasound device captured by the processing device), an array of values that is the same size as the inputted image, where each pixel in the array consists of a probability that that pixel is where the tip of the ultrasound image is located in the inputted image. The processing device may then predict that the pixel having the highest probability represents the location of the tip of the ultrasound image and output the horizontal and vertical coordinates of this pixel.

In some embodiments, a statistical model may be trained to use regression to determine the position of the ultrasound device relative to the processing device. Multiple images of the ultrasound device may be inputted to the statistical model as training input data. As training output data, each input image may be manually labeled with two numbers, namely the horizontal and vertical pixel coordinates of the tip of the ultrasound device (namely, the end of the ultrasound device opposite the sensor portion) in the image. Based on this training data, the statistical model may learn to output, based on an inputted image (e.g., a frame of the video of the ultrasound device captured by the processing device), the horizontal and vertical pixel coordinates of the tip of the ultrasound device in the image.

In some embodiments, a statistical model may be trained as a segmentation model to determine the position of the ultrasound device relative to the processing device. Multiple images of the ultrasound device may be inputted to the statistical model as training input data. As training output data, a segmentation mask may be inputted to the statistical model, where the segmentation mask is an array of values equal in size to the image, and pixels corresponding to locations within the ultrasound device in the image are manually set to 1 and other pixels are set to 0. Based on this training data, the statistical model may learn to output, based on an inputted image (e.g., a frame of the video of the ultrasound device captured by the processing device), a segmentation mask where each pixel has a value representing the probability that the pixel corresponds to a location within the ultrasound device in the image (values closer to 1) or outside the ultrasound device (values closer to 0). Horizontal and vertical pixel coordinates representing a single location of the ultrasound device in the image may then be derived (e.g., using averaging or some other method for deriving a single value from multiple values) from this segmentation mask.

In some embodiments, to determine the depth (z-direction) of the tip of the ultrasound device relative to the processing device, the processing device may use a depth camera on the processing device. For example, the depth camera may use disparity maps or structure light cameras. Such cameras may be considered stereo cameras in that they may use two cameras at different locations on the processing device that simultaneously capture two images, and the disparity between the two images may be used to determine the depth of the tip of the ultrasound device depicted in both images. In some embodiments, a time-of-flight camera may be used to determine the depth of the tip of the ultrasound device. In some embodiments, the processing device may use such depth cameras to determine the depth of the tip of the ultrasound device, and use a statistical model to determine horizontal and vertical coordinates of the tip of the ultrasound device in video captured with just one camera, as described above. However, in other embodiments, a statistical model may be trained to determine the depth from the image captured with just one camera. To train the statistical model, multiple images may be labeled with the depth of the tip of the ultrasound device in each image, where the depth may be determined using any method such as a depth camera. Thus, the processing device may use a statistical model to determine horizontal and vertical coordinates of the tip of the ultrasound device as well as the depth of the tip based on video captured with just one camera. In some embodiments, the processing device may assume a predefined depth as the depth of the tip of the ultrasound device relative to the processing device.

Using camera intrinsics (e.g., focal lengths, skew coefficient, and principal points), the processing device may convert the horizontal and vertical pixel coordinates of the tip of the ultrasound device into the horizontal (x-direction) and vertical (y-direction) distance of the tip of the ultrasound device relative to the processing device (more precisely, relative to the camera of the processing device). Note that the processing device may also use the depth to determine the horizontal and vertical distance. The distances of the tip of the ultrasound device relative to the processing device in the x-, y-, and z-directions may be considered the position of the tip of the ultrasound device relative to the processing device. It should be appreciated that as an alternative to the tip of the ultrasound device, any feature on the ultrasound device may be used instead.

In some embodiments, an auxiliary marker on the ultrasound device may be used to determine the distances of that feature relative to the processing device in the x-, y-, and z-directions based on video of the ultrasound device captured by the processing device, using pose estimation techniques and without using statistical models. For example, the auxiliary marker may be a marker conforming to the ArUco library, a color band, or some feature that is part of the ultrasound device itself.

In some embodiments, the processing device may determine, based on motion and/or orientation data from the processing device and motion and/or orientation data from the ultrasound device, an orientation of the ultrasound device relative to the processing device. The motion and/or orientation data from the ultrasound device may be collected by a motion and/or orientation sensor on the ultrasound device. The motion and/or orientation data from the processing device may be collected by a motion and/or orientation sensor on the processing device. The motion and/or orientation data may include data regarding acceleration, data regarding angular velocity, and/or data regarding magnetic force (which, due to the magnetic field of the earth, may be indicative of orientation relative to the earth). One or more accelerometers, gyroscopes, and/or magnetometers in each device may be used to generate the motion and/or orientation data. Depending on the sensors used to generate the motion and/or orientation data, the motion and/or orientation data may describe three degrees of freedom, six degrees of freedom, or nine degrees of freedom for the ultrasound device. Using sensor fusion techniques (e.g., based on Kalman filters, complimentary filters, and/or algorithms such as the Madgwick algorithm), the motion and/or orientation data may be used to generate the roll, pitch, and yaw angles of the device relative to a coordinate system defined by the directions of the local gravitational acceleration and the local magnetic field. If the roll, pitch, and yaw angles of each device are described by a rotation matrix, then multiplying the rotation matrix of the processing device by the inverse of the rotation matrix of the ultrasound device may produce a matrix describing the orientation (namely, the roll, pitch, and yaw angles) of the ultrasound device relative to the processing device.

In some embodiments, other methods may be used to determine the orientation of the ultrasound device relative to the processing device. For example, a statistical model may be trained to locate three different features of the ultrasound device in the video of the ultrasound device captured by the processing device (e.g., using methods described above for locating the tip of the ultrasound device in an image), from which the orientation of the ultrasound device may be uniquely determined. In some embodiments, a statistical model may be trained to determine, from an image or video of the ultrasound device captured by the processing device, the orientation of the ultrasound device relative to the processing device using regression. The statistical model may be trained on training input and output data, where the training input data is an image of the ultrasound device captured by the processing device and the output data consists of three numbers, namely the roll, pitch, and yaw angles (in other words, the orientation) of the ultrasound device relative to the processing device. The roll, pitch, and yaw angles for the output data may be determined from the motion and/or orientation sensor on the ultrasound device (e.g., the motion and/or orientation sensor 106) and the motion and/or orientation sensor on the processing device (e.g., the motion and/or orientation sensor 118) using the method described above. In some embodiments, the orientation of the ultrasound device relative to the earth may be determined up to the angle of the ultrasound device relative to the axis of gravity based on motion and/or orientation sensors on the ultrasound device (e.g., based on the accelerometer and/or gyroscope), and the orientation of the ultrasound device around the axis of gravity may be determined from video of the ultrasound device captured by the processing device (rather than, for example, a magnetometer of the ultrasound device) using a statistical model. The statistical model may be trained on images labeled with the angle around the axis of gravity, where the label is derived from magnetometer data. In some embodiments, methods described for determining orientation using the video of the ultrasound device and using motion and/or orientation sensors may both be used and combined into a single prediction that may be more reliable than if only one method were used.

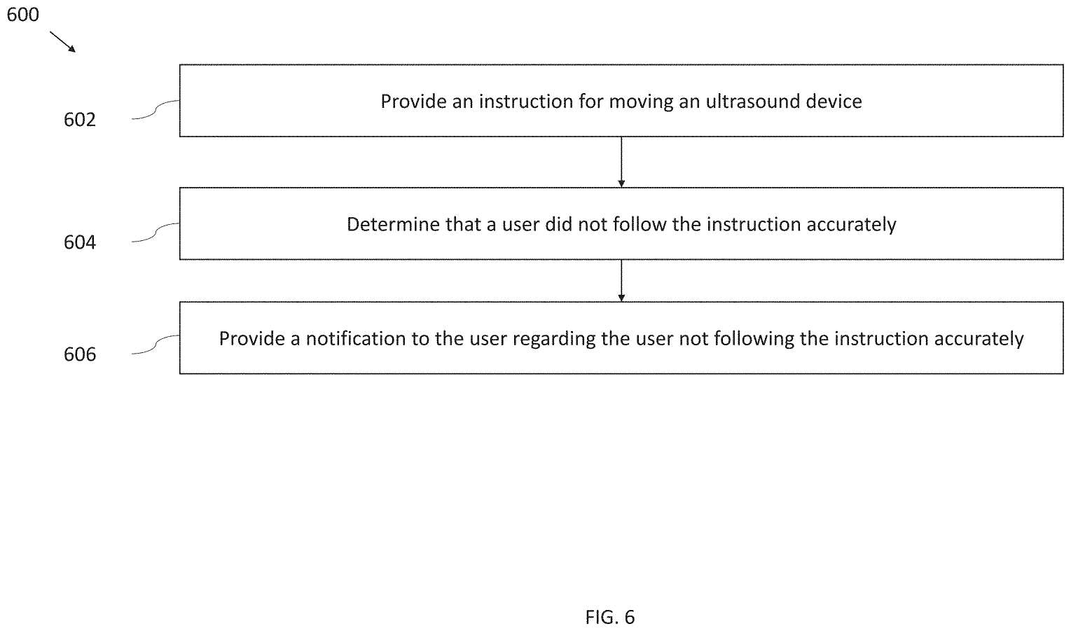

FIG. 6 is another flow diagram of a process 600 for how a processing device may incorporate how a user has previously moved an ultrasound device when providing instructions for moving the ultrasound device, in accordance with certain embodiments described herein. The process 600 is performed by a processing device in operative communication with an ultrasound device. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself may perform the process 600.

In act 602, the processing device provides an instruction for moving the ultrasound device. Further description of providing instructions may be found with reference to act 502. The process 600 proceeds from act 602 to act 604.

In act 604, the processing device determines that a user did not follow the instruction provided in act 602 accurately. Further description of such a determination may be found with reference to act 504. The process 600 proceeds from act 604 to act 606.

In act 606, based on determining that the user did not follow the instruction accurately, the processing device provides a notification to the user regarding the user not following the instruction accurately. In some embodiments, the notification may communicate to the user that the user should carefully follow instructions. As one example, the notification may include text communicating to the user that the user should carefully follow instructions. As another example, the notification may include highlighting graphical directions (e.g., arrows). In some embodiments, the notification may indicate to the user (e.g., through text) that the user may access a help page to obtain a refresher on how to use the processing device.

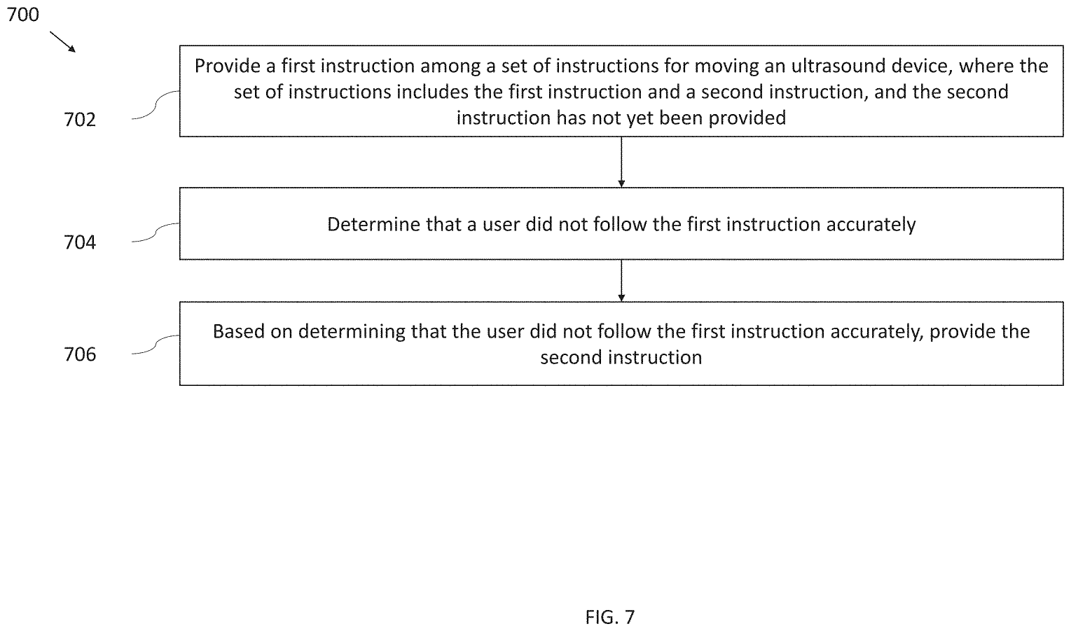

FIG. 7 is another flow diagram of a process 700 for how a processing device may incorporate how a user has previously moved an ultrasound device when providing instructions for moving the ultrasound device, in accordance with certain embodiments described herein. The process 700 is performed by a processing device in operative communication with an ultrasound device. The processing device may be, for example, a mobile phone, tablet, or laptop in operative communication with an ultrasound device. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). In some embodiments, the ultrasound device itself may perform the process 700.

In act 702, the processing device provides a first instruction. The first instruction is one instruction among a set of instructions for moving an ultrasound device. The set of instructions further includes a second instruction that has not yet been provided. For example, the set of instructions may include a first instruction to tilt the ultrasound device and a second instruction to translate the ultrasound device, in order to move the ultrasound device from a current position to a target position. Further description of instructions may be found with reference to act 502. The process 700 proceeds from act 702 to act 704.

In act 704, the processing device determines that the user did not follow the first instruction provided in act 702 accurately. Further description of such a determination may be found with reference to act 504. The process 700 proceeds from act 704 to act 706.

In act 706, based on determining that the user did not follow the first instruction accurately, the processing device provides the second instruction. In other words, the processing device may cease to provide the first instruction before the user has accurately followed the first instruction. There may be multiple paths for moving an ultrasound device from a given location to another given location. For example, if one path from a current position to a target position includes multiple steps of translations, rotations, and/or tilts in a particular order, another path may include the steps in a different order. In other words, steps for moving an ultrasound device from one position to another may be commutative. If the processing device is providing one instruction among multiple instructions in a set, and the user does not accurately follow that instruction, the processing device may switch to provide another instruction in the set. Thus, the processing device may effectively delay the instruction it was previously providing. If the user accurately follows this other instruction, then the user may have moved the ultrasound device closer to the target position than if the processing device had continued to provide the instruction that the user was not following accurately. Once the user has moved the ultrasound device closer to the target position, the processing device may again provide the instruction that the user did not follow accurately, or the ultrasound device may already be sufficiently close to the target position (e.g., close enough to collect the desired anatomical view). In other words, rather than the processing device getting “stuck” providing an instruction that the user is not following accurately, the processing device may provide another instructions to try to get the ultrasound device closer to the target position.

In the above example, the set of instructions for moving the ultrasound device to the target position included a first instruction to tilt the ultrasound device and a second instruction to translate the ultrasound device. The processing device was providing the first instruction before providing the second instruction. However, upon determining that the user did not follow the first instruction to tilt the ultrasound device accurately, the processing device switches to providing the second instruction to translate the ultrasound device. After providing the second instruction to translate the ultrasound device, the processing device may again provide the instruction to tilt the ultrasound device. Alternatively, the ultrasound device may already be sufficiently close to the target position, thus obviating the need to provide the first instruction to tilt the ultrasound device.

In some embodiments, at act 702, the processing device may provide a first type of instruction among a set of multiple types of instructions (e.g., translating, tilting, and rotating) for moving an ultrasound device, where the set of instructions includes the first type of instruction and a second type of instruction, and the second type of instruction has not yet been provided. At act 704, the processing device may determine that a user did not follow the first type of instruction accurately. At act 706, based on determining that the user did not follow the first type of instruction accurately, the processing device may provide the second type of instruction. Thus, if the user is not following a particular type of instruction accurately, the processing device may switch to providing another type of instruction.

It may be evident that the process 700 is being performed, for example, if the processing device ceases to provide one instruction (or one type of instruction) when the user does not follow that instruction (or type of instruction) accurately, and begins to provide another instruction (or another type of instruction).

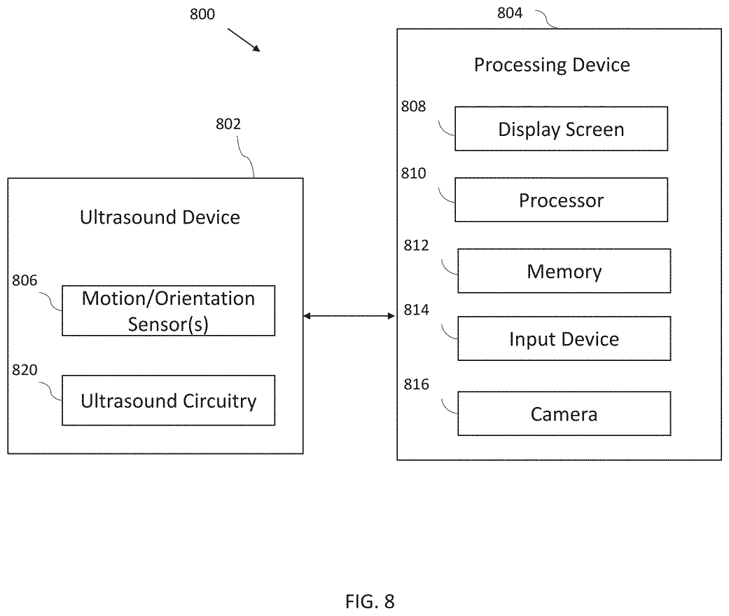

FIG. 8 illustrates a schematic block diagram of an example ultrasound system 800 upon which various aspects of the technology described herein may be practiced. The ultrasound system 800 includes an ultrasound device 802 and a processing device 804. The ultrasound device 802 may be the same as the ultrasound device 100 and/or the ultrasound device discussed with reference to the processes 500-700. The processing device 804 may be the same as the processing device discussed with reference to FIG. 1 and the processes 500-700.

The ultrasound device 802 includes a motion and/or orientation sensor(s) 806 and ultrasound circuitry 820. The processing device 804 includes a camera 816, a display screen 808, a processor 810, a memory 812, and an input device 814. The processing device 804 is in wired (e.g., through a lightning connector or a mini-USB connector) and/or wireless communication (e.g., using BLUETOOTH, ZIGBEE, and/or WiFi wireless protocols) with the ultrasound device 802.

The ultrasound device 802 may be configured to generate ultrasound data that may be employed to generate an ultrasound image. The ultrasound device 802 may be constructed in any of a variety of ways. In some embodiments, the ultrasound device 802 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound circuitry 820 may be configured to generate the ultrasound data. The ultrasound circuitry 820 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 820 (e.g., transmit circuitry, receive circuitry, control circuitry, power management circuitry, and processing circuitry) to form a monolithic ultrasound device. The ultrasound device 802 may transmit ultrasound data and/or ultrasound images to the processing device 804 over a wired (e.g., through a lightning connector or a mini-USB connector) and/or wireless (e.g., using BLUETOOTH, ZIGBEE, and/or WiFi wireless protocols) communication link.

The motion and/or orientation sensor(s) 806 may be configured to generate motion and/or orientation data regarding the ultrasound device 802. For example, the motion and/or orientation sensor(s) 806 may be configured to generate data regarding acceleration of the ultrasound device 802, data regarding angular velocity of the ultrasound device 802, and/or data regarding magnetic force acting on the ultrasound device 802 due to the local magnetic field, which in many cases is simply the field of the earth. The motion and/or orientation sensor(s) 806 may include an accelerometer, a gyroscope, and/or a magnetometer. Depending on the sensors present in the motion and/or orientation sensor(s) 806, the motion and/or orientation data generated by the motion and/or orientation sensor(s) 806 may describe three degrees of freedom, six degrees of freedom, or nine degrees of freedom for the ultrasound device 802. For example, the motion and/or orientation sensor(s) 806 may include an accelerometer, a gyroscope, and/or magnetometer. Each of these types of sensors may describe three degrees of freedom. If the motion and/or orientation sensor(s) 806 includes one of these sensors, the motion and/or orientation sensor(s) 806 may describe three degrees of freedom. If the motion and/or orientation sensor(s) 806 includes two of these sensors, the motion and/or orientation sensor(s) 806 may describe two degrees of freedom. If the motion and/or orientation sensor(s) 806 includes three of these sensors, the motion and/or orientation sensor(s) 806 may describe nine degrees of freedom. The ultrasound device 802 may transmit data to the processing device 804 over a wired (e.g., through a lightning connector or a mini-USB connector) and/or wireless (e.g., using BLUETOOTH, ZIGBEE, and/or WiFi wireless protocols) communication link.

Referring now to the processing device 804, the processor 810 may include specially-programmed and/or special-purpose hardware such as an application-specific integrated circuit (ASIC). For example, the processor 810 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network. The processing device 804 may be configured to process the ultrasound data received from the ultrasound device 802 to generate ultrasound images for display on the display screen 808. The processing may be performed by, for example, the processor 810. The processor 810 may also be adapted to control the acquisition of ultrasound data with the ultrasound device 802. The ultrasound data may be processed in real-time during a scanning session as the echo signals are received. In some embodiments, the displayed ultrasound image may be updated a rate of at least 8 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

The processing device 804 may be configured to perform certain of the processes described herein using the processor 810 (e.g., one or more computer hardware processors) and one or more articles of manufacture that include non-transitory computer-readable storage media such as the memory 812. The processor 810 may control writing data to and reading data from the memory 812 in any suitable manner. To perform certain of the processes described herein, the processor 810 may execute one or more processor-executable instructions stored in one or more non-transitory computer-readable storage media (e.g., the memory 812), which may serve as non-transitory computer-readable storage media storing processor-executable instructions for execution by the processor 810. The camera 816 may be configured to detect light (e.g., visible light) to form an image or a video. The display screen 808 may be configured to display images and/or videos, and may be, for example, a liquid crystal display (LCD), a plasma display, and/or an organic light emitting diode (OLED) display on the processing device 804. The input device 814 may include one or more devices capable of receiving input from a user and transmitting the input to the processor 810. For example, the input device 814 may include a keyboard, a mouse, a microphone, touch-enabled sensors on the display screen 808, and/or a microphone. The display screen 808, the input device 814, the camera 816, and the speaker 806 may be communicatively coupled to the processor 810 and/or under the control of the processor 810.

It should be appreciated that the processing device 804 may be implemented in any of a variety of ways. For example, the processing device 804 may be implemented as a handheld device such as a mobile smartphone or a tablet. Thereby, a user of the ultrasound device 802 may be able to operate the ultrasound device 802 with one hand and hold the processing device 804 with another hand. In other examples, the processing device 804 may be implemented as a portable device that is not a handheld device, such as a laptop. In yet other examples, the processing device 804 may be implemented as a stationary device such as a desktop computer. For further description of ultrasound devices and systems, see U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application).

FIG. 8 should be understood to be non-limiting. For example, the ultrasound device 802 and/or the processing device 804 may include fewer or more components than shown.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Various inventive concepts may be embodied as one or more processes, of which an example has been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus, comprising a processing device in operative communication with an ultrasound device, the processing device configured to:
provide a first instruction for moving the ultrasound device;
determine a difference between the first instruction and a movement of the ultrasound device;
determine, based on the difference between the first instruction and the movement of the ultrasound device, a second instruction for moving the ultrasound device; and
provide the second instruction for moving the ultrasound device.

2. The apparatus of claim 1, wherein the processing device is configured, when determining the difference between the first instruction and the movement of the ultrasound device, to determine the movement of the ultrasound device by determining motion and/or orientation of the ultrasound device relative to the processing device.

3. The apparatus of claim 1, wherein the processing device is configured, when determining the difference between the first instruction and the movement of the ultrasound device, to determine the movement of the ultrasound device by using one or more of images/video from the processing device, motion and/or orientation data from the processing device, and motion and/or orientation data from the ultrasound device to determine the movement of the ultrasound device relative to the processing device.

4. The apparatus of claim 1, wherein the first instruction indicates a direction for moving the ultrasound device, the movement data describes a direction that the ultrasound device has moved, and the processing device is configured, when determining the difference between the first instruction and the movement of the ultrasound device, to determine a difference between the direction for moving the ultrasound device and the direction that the ultrasound device has moved.

5. The apparatus of claim 1, wherein the processing device is configured, when determining the second instruction for moving the ultrasound device, to determine the second instruction such that the second instruction compensates for the difference between the first instruction and the movement of the ultrasound device.

6. The apparatus of claim 1, wherein the processing device is configured, when determining the second instruction for moving the ultrasound device, to subtract the difference between the first instruction and the movement of the ultrasound device from a direction in which the processing device determines the ultrasound device should be moved.

7. The apparatus of claim 1, wherein the processing device is configured to provide the first instruction and the second instruction as part of a single scan.

8. The apparatus of claim 1, wherein the processing device is configured to provide the first instruction and the second instruction for moving the ultrasound device to a target pose.

9. The apparatus of claim 1, wherein the processing device is configured to provide the first instruction for moving the ultrasound device to a first target pose, and to provide the second instruction for moving the ultrasound device to a second target pose.

10. The apparatus of claim 9, wherein the first target pose is a pose where the ultrasound device can collect ultrasound data from one anatomical region, and the second target pose is a pose where the ultrasound device can collect ultrasound data from a second anatomical region.

11. The apparatus of claim 10, wherein the first and second anatomical regions are anatomical regions scanned as a part of an imaging protocol.

12. The apparatus of claim 1, wherein the processing device is configured to provide the first instruction and the second instruction as part of different scans.

13. The apparatus of claim 1, wherein the processing device is configured to store in memory the difference between the first instruction and the movement of the ultrasound device from scan to scan and use that difference for providing the second instruction in a subsequent scan.

14. The apparatus of claim 1, wherein the processing device is configured to compute an average of differences between instructions provided and subsequent movements of the ultrasound device across multiple scans, and to use the average of differences to provide the second instruction.

15. An apparatus, comprising a processing device in operative communication with an ultrasound device, the processing device configured to:
provide an instruction for moving the ultrasound device;
determine that a user did not follow the instruction accurately; and
based on determining that the user did not follow the instruction accurately, provide a notification to the user regarding the user not following the instruction accurately.

16. The apparatus of claim 15, wherein the notification communicates to the user that the user should carefully follow instructions.

17. The apparatus of claim 15, wherein the processing device is configured, when providing the notification, to highlight graphical directions.

18. The apparatus of claim 15, wherein the notification indicates to the user that the user can access a help page to obtain a refresher on how to use the processing device.

19. An apparatus, comprising a processing device in operative communication with an ultrasound device, the processing device configured to:
provide a first instruction among a set of instructions for moving the ultrasound device, where the set of instructions includes the first instruction and a second instruction, and the second instruction has not yet been provided;
determine that a user did not follow the first instruction accurately; and
based on determining that the user did not follow the first instruction accurately, provide the second instruction.

20. The apparatus of claim 19, wherein the processing device is configured to cease to provide the first instruction before the user has accurately followed the first instruction.
